(19) United States
(12) Patent Application Publication
(10) Pub. No.: US 2006/0287528 A1
(43) Pub. Date: Dec. 21, 2006

(54) Novel crystalline forms of a phosphoric acid salt of a dipeptidyl peptidase-iv inhibitor

(76) Inventors: Robert M. Wenslow, 126 EAST LINCOLN AVENUE RAHWAY NJ (US); Joseph D. Armstrong III, (US); Alex M. Chen, (US); Stephen Cypes, (US); Russell R. Ferlita, (US); Karl Hansen, (US); Christopher Lindemann, (US); Evangelia Spartalis, (US)

Correspondence Address: MERCK AND CO., INC, P O BOX 2000, RAHWAY, NJ, 07065-0907, US

(21) Appl. No.: 10/569,566

(22) PCT Filed: Aug. 27, 2004
(86) PCT No.: PCT/US04/27983

§ 371 Date: Feb. 27, 2006

Related U.S. Application Data
(60) Provisional application No. 60/499,629, filed on Sep. 2, 2003.

(51) Int. Cl. C07D 487/04

(52) U.S. Cl. 544/350

(57) ABSTRACT

The present invention relates to crystalline anhydrate polymorphs of the dihydrogenphosphate salt of (2R)4-oxo-4-[3-(trifluoromethyl)-5,6-dihydro[1,2,4]triazolo[4,3-α]pyrazin-7(8H) yl]-1-(2,4,5-trifluorophenyl)butan-2-amine as well as a process for their preparation, pharmaceutical compositions containing these novel forms, and methods of use of the novel forms and pharmaceutical compositions for the treatment of diabetes, obesity, and high blood pressure. The invention also concerns novel crystalline solvates of the dihydrogenphosphate salt of (2R)-4-oxo-4-[3-(trifluoromethyl)-5,6-dihydro[1,2,4]tria-zolo [4,3-α]pyrazin-7(8H)-yl]-1-(2,4,5-trifluorophenyl)butan-2-amine as well as a crystalline desolvated polymorph and their use for the preparation of the anhydrate polymorphs of the present invention.

### FIELD OF THE INVENTION

The present invention relates to novel crystalline forms of a dihydrogenphosphate salt of a dipeptidyl peptidase-IV inhibitor. More particularly, the invention relates to novel crystalline solvates and anhydrates of the dihydrogenphosphate salt of (2R)-4-oxo4-[3-(trifluoromethyl)-5,6-dihydro[1,2,4]triazolo[4,3-α]pyrazin-7(8H)-yl]-1-(2,4,5-trifluorophenyl)butan-2-amine, which is a potent inhibitor of dipeptidyl peptidase-IV (DPP-IV). These novel crystalline forms of the DPP-IV inhibitor are useful for the preparation of pharmaceutical compositions containing the inhibitor which are useful for the treatment and prevention of diseases and conditions for which an inhibitor of dipeptidyl peptidase-IV is indicated, in particular Type 2 diabetes, hyperglycemia, insulin resistance, obesity, and high blood pressure. The invention further concerns pharmaceutical compositions comprising the novel crystalline dihydrogenphosphate salt anhydrate polymorphic forms of the present invention; processes for preparing the dihydrogenphosphate salt solvates and anhydrates and their pharmaceutical compositions; and methods of treating conditions for which a DPP-IV inhibitor is indicated comprising administering a composition of the present invention.

### BACKGROUND OF THE INVENTION

Inhibition of dipeptidyl peptidase-IV (DPP-IV), an enzyme that inactivates both glucose-dependent insulinotropic peptide (GIP) and glucagon-like peptide 1 (GLP-1), represents a novel approach to the treatment and prevention of Type 2 diabetes, also known as non-insulin dependent diabetes mellitus (NIDDM). The therapeutic potential of DPP-IV inhibitors for the treatment of Type 2 diabetes has been reviewed: C. F. Deacon and J. J. Holst, “Dipeptidyl peptidase IV inhibition as an approach to the treatment and prevention of Type 2 diabetes: a historical perspective,” Biochem Biophys. Res. Commun., 294: 1-4 (2000); K. Augustyns, et al., “Dipeptidyl peptidase IV inhibitors as new therapeutic agents for the treatment of Type 2 diabetes,” Exp. Opin. Ther. Patents, 13: 499-510 (2003); and D. J. Drucker, “Therapeutic potential of dipeptidyl peptidase IV inhibitors for the treatment of Type 2 diabetes,” Exp. Opin. Investig. Drugs, 12: 87-100 (2003).

WO 03/004498 (published 16 Jan. 2003) and U.S. Pat. No. 6,699,871 (issued Mar. 2, 2004), both assigned to Merck & Co., describe a class of beta-amino tetrahydrotriazolo[4,3-α]pyrazines, which are potent inhibitors of DPP-IV and therefore useful for the treatment of Type 2 diabetes. Specifically disclosed in WO 03/004498 is (2R)-4-oxo-4-[3-(trifluoromethyl)-5,6-dihydro[1,2,4]triazolo[4,3-α]pyrazin-7(8H)-yl]-1-(2,4,5-trifluorophenyl)butan-2-amine.

However, there is no disclosure in the above references of the newly discovered crystalline solvates and anhydrates of the dihydrogenphosphate salt of (2R)4-oxo-4-[3-(trifluoromethyl)-5,6-dihydro[1,2,4]triazolo[4,3-α]pyrazin-7(8H)-yl]-1-(2,4,5-trifluorophenyl)butan-2-amine of structural formula I below (hereinafter referred to as Compound I).

### SUMMARY OF THE INVENTION

The present invention is concerned with novel crystalline solvates and anhydrates of the dihydrogenphosphate salt of the dipeptidyl peptidase-IV (DPP-IV) inhibitor (2R)-4-oxo-4-[3-(trifluoromethyl)-5,6-dihydro[1,2,4]triazolo[4,3-α]pyrazin-7(8H)-yl]-1-(2,4,5-trifluorophenyl)butan-2-amine of structural formula I (Compound I). The crystalline solvates and anhydrates of the present invention have advantages in the preparation of pharmaceutical compositions of the dihydrogenphosphate salt of (2R)-4-oxo-4-[3-(trifluoromethyl)-5,6-dihydro[1,2,4]triazolo[4,3-α]pyrazin-7(8H)-yl]-1-(2,4,5-trifluorophenyl)butan-2-amine, such as ease of processing, handling, and dosing. In particular, they exhibit improved physicochemical properties, such as solubility, stability to stress, and rate of dissolution, rendering them particularly suitable for the manufacture of various pharmaceutical dosage forms. The invention also concerns pharmaceutical compositions containing the novel anhydrate polymorphs; processes for the preparation of these solvates and anhydrates and their pharmaceutical compositions; and methods for using them for the prevention or treatment of Type 2 diabetes, hyperglycemia, insulin resistance, obesity, and high blood pressure.

### BRIEF DESCRIPTION OF THE FIGURES

FIG. 1 is a characteristic X-ray diffraction pattern of the crystalline anhydrate Form I of Compound I.

FIG. 2 is a carbon-13 cross-polarization magic-angle spinning (CPMAS) nuclear magnetic resonance (NMR) spectrum of the crystalline anhydrate Form I of Compound I.

FIG. 3 is a fluorine-19 magic-angle spinning (MAS) nuclear magnetic resonance (NMR) spectrum of the crystalline anhydrate Form I of Compound I.

FIG. 4 is a typical DSC curve of the crystalline anhydrate Form I of Compound I.

FIG. 5 is a typical thermogravimetric (TG) curve of the crystalline anhydrate Form I of Compound I.

FIG. 6 is a characteristic X-ray diffraction pattern of the crystalline desolvated anhydrate Form II of Compound I.

FIG. 7 is a carbon-13 cross-polarization magic-angle spinning (CPMAS) nuclear magnetic resonance (NMR) spectrum of the crystalline desolvated anhydrate Form II of Compound I.

FIG. 8 is a fluorine-19 magic-angle spinning (MAS) nuclear magnetic resonance (NMR) spectrum of the crystalline desolvated anhydrate Form II of Compound I.

FIG. 9 is a typical DSC curve of the crystalline desolvated anhydrate Form II of Compound I.

FIG. 10 is a typical TG curve of the crystalline desolvated anhydrate Form II of Compound I.

FIG. 11 is a characteristic X-ray diffraction pattern of the crystalline anhydrate Form III of Compound I.

FIG. 12 is a carbon-13 cross-polarization magic-angle spinning (CPMAS) nuclear magnetic resonance (NMR) spectrum of the crystalline anhydrate Form III of Compound I.

FIG. 13 is a fluorine-19 magic-angle spinning (MAS) nuclear magnetic resonance (NMR) spectrum of the crystalline anhydrate Form III of Compound I.

FIG. 14 is a typical DSC curve of the crystalline anhydrate Form III of Compound I.

FIG. 15 is a typical TG curve of the crystalline anhydrate Form III of Compound I.

FIG. 16 is a characteristic X-ray diffraction pattern of the crystalline ethanol solvate of Compound I.

FIG. 17 is a carbon-13 cross-polarization magic-angle spinning (CPMAS) nuclear magnetic resonance (NMR) spectrum of the crystalline ethanol solvate of Compound I.

FIG. 18 is a fluorine-19 magic-angle spinning (MAS) nuclear magnetic resonance (NMR) spectrum of the crystalline ethanol solvate of Compound I.

FIG. 19 is a typical DSC curve of the crystalline ethanol solvate of Compound I.

FIG. 20 is a typical TG curve of the crystalline ethanol solvate of Compound I.

### DETAILED DESCRIPTION OF THE INVENTION

This invention provides novel crystalline solvates and anhydrates of the dihydrogenphosphate salt of (2R)-4-oxo-4-[3-trifluoromethyl)-5,6-dihydro[1,2,4]triazolo[4,3-α]pyrazin-7(8H)-yl]-1-(2,4,5-trifluorophenyl)butan-2-amine of structural formula I (Compound I):

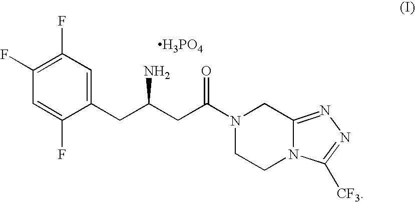

In one embodiment the solvate is a C₁₋₄ alkanolate of Compound I. In a class of this embodiment the C₁₋₄ alkanolate is a methanolate, ethanolate, 1-propanolate, or 2-propanolate. In another embodiment the solvate comprises an organic solvent such as acetone or acetonitrile. The crystalline solvates are useful for the preparation of the crystalline desolvated anhydrate Form II which converts spontaneously into crystalline anhydrate Form I or Form III or a mixture thereof, the composition of the mixture being dependent upon the conditions of treatment or storage. Anhydrate Forms I and III represent stable desolvated anhydrates of Compound I.

The present invention also provides a novel crystalline desolvated anhydrate Form II of Compound I which is obtained from the crystalline solvates of Compound I of the present invention.

The present invention also provides novel crystalline anhydrate Forms I and III of Compound I and mixtures thereof.

A further embodiment of the present invention provides the Compound I drug substance that comprises the crystalline anhydrate Form I or III or a mixture thereof in a detectable amount. By “drug substance” is meant the active pharmaceutical ingredient (API). The amount of crystalline anhydrate Form I or III or mixture thereof in the drug substance can be quantified by the use of physical methods such as X-ray powder diffraction (XRPD), solid-state fluorine-19 magic-angle spinning (MAS) nuclear magnetic resonance spectroscopy, solid-state carbon-13 cross-polarization magic-angle spinning (CPMAS) nuclear magnetic resonance spectroscopy, solid state Fourier-transform infrared spectroscopy, and Raman spectroscopy. In a class of this embodiment, about 5% to about 100% by weight of the crystalline anhydrate Form I or III or mixture thereof is present in the drug substance. In a second class of this embodiment, about 10% to about 100% by weight of the crystalline anhydrate Form I or III or mixture thereof is present in the drug substance. In a third class of this embodiment, about 25% to about 100% by weight of the crystalline anhydrate Form I or III or mixture thereof is present in the drug substance. In a fourth class of this embodiment, about 50% to about 100% by weight of the crystalline anhydrate Form I or III or mixture thereof is present in the drug substance. In a fifth class of this embodiment, about 75% to about 100% by weight of the crystalline anhydrate Form I or III or mixture thereof is present in the drug substance. In a sixth class of this embodiment, substantially all of the Compound I drug substance is the crystalline anhydrate Form I or III or mixture thereof, i.e., the Compound I drug substance is substantially phase pure anhydrate Form I or III or a mixture thereof.

The crystalline solvates of the present invention are useful for the preparation of the crystalline anhydrate Forms I and III and mixtures thereof. The crystalline solvates are desolvated to afford the intermediate desolvated anhydrate Form II which converts into anhydrate Form I or Form III or a mixture thereof upon heating at 45° C. for about 2 h.

Another aspect of the present invention provides a method for the prevention or treatment of clinical conditions for which an inhibitor of DPP-IV is indicated, which method comprises administering to a patient in need of such prevention or treatment a prophylactically or therapeutically effective amount of the crystalline anhydrate Form I or III or a mixture thereof of Compound I. Such clinical conditions include diabetes, in particular Type 2 diabetes, hyperglycemia, insulin resistance, obesity, and high blood pressure.

The present invention also provides for the use of the crystalline anhydrate Form I or III or a mixture thereof of the present invention in the manufacture of a medicament for the prevention or treatment of clinical conditions for which an inhibitor of DPP-IV is indicated, in particular, Type 2 diabetes, hyperglycemia, insulin resistance, obesity, and high blood pressure. In one embodiment the clinical condition is Type 2 diabetes.

Another aspect of the present invention provides the crystalline anhydrate Form I or Form III or a mixture thereof for use in the treatment of clinical conditions for which an inhibitor of DPP-IV is indicated, in particular, Type 2 diabetes, hyperglycemia, insulin resistance, obesity, and high blood pressure. In one embodiment of this aspect the clinical condition is Type 2 diabetes.

The present invention also provides pharmaceutical compositions comprising the crystalline anhydrate Form I or III or a mixture thereof, in association with one or more pharmaceutically acceptable carriers or excipients. In one embodiment the pharmaceutical composition comprises a prophylactically or therapeutically effective amount of the active pharmaceutical ingredient (API) in admixture with pharmaceutically acceptable excipients wherein the API comprises a detectable amount of the crystalline anhydrate Form I or III or a mixture thereof of the present invention. In a second embodiment the pharmaceutical composition comprises a prophylactically or therapeutically effective amount of the API in admixture with pharmaceutically acceptable excipients wherein the API comprises about 5% to about 100% by weight of the crystalline anhydrate Form I or III or a mixture thereof of the present invention. In a class of this second embodiment, the API in such compositions comprises about 10% to about 100% by weight of the crystalline anhydrate Form I or III or a mixture thereof. In a second class of this embodiment, the API in such compositions comprises about 25% to about 100% by weight of the crystalline anhydrate Form I or III or a mixture thereof. In a third class of this embodiment, the API in such compositions comprises about 50% to about 100% by weight of the crystalline anhydrate Form I or III or a mixture thereof. In a fourth class of this embodiment, the API in such compositions comprises about 75% to about 100% by weight of the crystalline anhydrate Form I or III or a mixture thereof. In a fifth class of this embodiment, substantially all of the API is the crystalline anhydrate Form I or III or a mixture thereof of Compound I, i.e., the API is substantially phase pure Compound I anhydrate Form I or III or a mixture thereof.

The compositions in accordance with the invention are suitably in unit dosage forms such as tablets, pills, capsules, powders, granules, sterile solutions or suspensions, metered aerosol or liquid sprays, drops, ampoules, auto-injector devices or suppositories. The compositions are intended for oral, parenteral, intranasal, sublingual, or rectal administration, or for administration by inhalation or insufflation. Formulation of the compositions according to the invention can conveniently be effected by methods known from the art, for example, as described in Remington's Pharmaceutical Sciences, 17^{th} ed., 1995.

The dosage regimen is selected in accordance with a variety of factors including type, species, age, weight, sex and medical condition of the patient; the severity of the condition to be treated; the route of administration; and the renal and hepatic function of the patient. An ordinarily skilled physician, veterinarian, or clinician can readily determine and prescribe the effective amount of the drug required to prevent, counter or arrest the progress of the condition.

Oral dosages of the present invention, when used for the indicated effects, will range between about 0.01 mg per kg of body weight per day (mg/kg/day) to about 100 mg/kg/day, preferably 0.01 to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100 and 500 milligrams of the API for the symptomatic adjustment of the dosage to the patient to be treated. A medicament typically contains from about 0.01 mg to about 500 mg of the API, preferably, from about 1 mg to about 200 mg of API. Intravenously, the most preferred doses will range from about 0.1 to about 10 mg/kg/minute during a constant rate infusion. Advantageously, the crystalline anhydrate forms of the present invention may be administered in a single daily dose, or the total daily dosage may be administered in divided doses of two, three or four times daily. Furthermore, the crystalline anhydrate forms of the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

In the methods of the present invention, the Compound I anhydrate Forms I and III or a mixture thereof herein described in detail can form the API, and are typically administered in admixture with suitable pharmaceutical diluents, excipients or carriers (collectively referred to herein as ‘carrier’ materials) suitably selected with respect to the intended form of administration, that is, oral tablets, capsules, elixirs, syrups and the like, and consistent with conventional pharmaceutical practices.

For instance, for oral administration in the form of a tablet or capsule, the active pharmaceutical ingredient can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral API can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The crystalline anhydrate Forms I and III or mixtures thereof of Compound I have been found to possess a high solubility in water, rendering them especially amenable to the preparation of formulations, in particular intranasal and intravenous formulations, which require relatively concentrated aqueous solutions of the API. The solubility of the crystalline Compound I anhydrate Form I or Form III or mixture thereof in water is greater than 120 mg/mL.

In a still further aspect, the present invention provides a method for the treatment and/or prevention of clinical conditions for which a DPP-IV inhibitor is indicated, which method comprises administering to a patient in need of such prevention or treatment a prophylactically or therapeutically effective amount of anhydrate Form I or III or a mixture thereof of the present invention or a pharmaceutical composition containing a prophylactically or therapeutically effective amount of anhydrate Form I or III or a mixture thereof.

The following non-limiting Examples are intended to illustrate the present invention and should not be construed as being limitations on the scope or spirit of the instant invention.

Compounds described herein may exist as tautomers such as keto-enol tautomers. The individual tautomers as well as mixtures thereof are encompassed with compounds of structural formula I.

The term “% enantiomeric excess” (abbreviated “ee”) shall mean the % major enantiomer less the % minor enantiomer. Thus, a 70% enantiomeric excess corresponds to formation of 85% of one enantiomer and 15% of the other. The term “enantiomeric excess” is synonymous with the term “optical purity.”

General Methods for Preparing Solvates of Compound I and the Desolvated Anhydrate Form II and for Preparing and Interconverting Between Anhydrate Forms I and III:

Compound I forms non-stoichiometric, isomorphous solvates with several organic solvents, such as methanol, ethanol, 1-propanol, 2-propanol, acetone, and acetonitrile. The various solvates of the present invention are isomorphic and exhibit similar X-ray powder diffraction patterns, F-19 solid-state NMR spectra, and DSC curves.

Solvates are prepared by contacting anhydrate Form I, II, or III, or mixtures thereof, with the solvating agent for about 5 min at about room temperature. Solvates will also result from the process of preparing the dihydrogenphosphate salt from free base in the presence of a solvating agent where the water activity is such that the solvate has a lower solubility than any of the other anhydrates or monohydrate. For example, the ethanol solvate can be formed by treating the free base with aqueous phosphoric acid in ethanol.

The ethanol solvate can be converted to desolvated anhydrate Form II by (a) drying with nitrogen flow over the sample for about 5 h at about 25° C. or (b) drying in vacuum for about 5 h at about 25° C.

Desolvated anhydrate Form II is metastable and converts to anhydrate Form I or Form III or mixtures thereof in about 2 h at about 45° C.

Anhydrate Form I can be converted into anhydrate Form III by (a) drying with physical agitation, (b) compaction, or (c) grinding. Anhydrate Form III can be converted into anhydrate Form I by heating at about 110° C. for about 30 min.

Mixtures of varying composition of anhydrate Forms I and III form upon grinding or compaction of Form I or mixtures thereof at room temperature, which results in the increased proportion of Form III in the mixture.

The anhydrate polymorphic Form I and Form III have an enantiotropic relationship, that is, one form is more stable at a lower temperature range, while the other is more stable at a higher temperature with a transition temperature of about 34° C. Anhydrate Form III is the low temperature stable form and is stable below about 34° C. Anhydrate Form I is the high temperature stable form and is, stable above about 34° C.

The anhydrate Forms I and III can be directly crystallized from a solvent that Compound I does not solvate with, such as isoamyl alcohol, at a water activity where the hydrate is not stable. Form III can be preferentially crystallized below about 34° C., and Form I can be preferentially crystallized above about 34° C.

General Conditions for Preferentially Crystallizing Anhydrate Form I:

In isoamyl alcohol (IAA)/water system at 40° C:
(1) crystallization from a mixture of compound I in IAA and water, such that the water concentration is below 3.4 weight percent;
(2) recovering the resultant solid phase; and
(3) removing the solvent therefrom.
   In IAA/water system at 60° C.:
1) crystallization from a mixture of compound I in IAA and water, such that the water concentration is below 4.5 weight percent;
(2) recovering the resultant solid phase; and
(3) removing the solvent therefrom.
   General Conditions for Preferentially Crystallizing Anhydrate Form III:

In isoamyl alcohol (IAA)/water system at 25° C.:
(1) crystallization from a mixture of compound I in IAA and water, such that the water concentration is below 2.7 weight percent;
(2) recovering the resultant solid phase; and
(3) removing the solvent therefrom.

### EXAMPLE 1

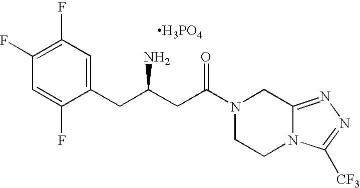 (2R)-4-oxo-4-[3-(trifluoromethyl)-5,6-dihydro[1,2,4]triazolo[4,3-α]pyrazin-7(8H)-yl]-1-(2,4,5-trifluorophenyl)butan-2-amine dihydrogenphosphate anhydrate Form I and Form III mixture
Preparation of 3-(trifluoromethyl)-5,6,7,8-tetrahydro[1,2,4]triazolo[4,3-α]pyrazine hydrochloride (1-4) 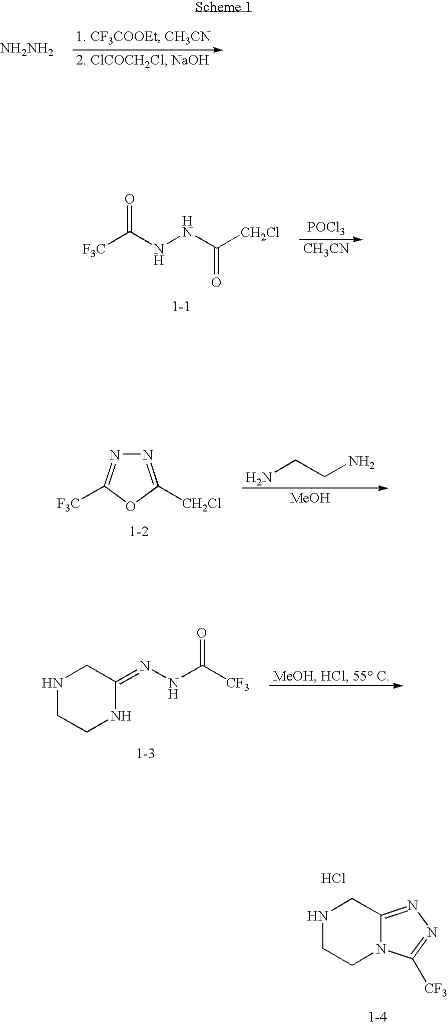
Step A: Preparation of Bishydrazide (1-1)

Hydrazine (20.1 g, 35 wt % in water, 0.22 mol) was mixed with 310 mL of acetonitrile. 31.5 g of ethyl trifluoroacetate (0.22 mol) was added over 60 min. The internal temperature was increased to 25° C. from 14° C. The resulting solution was aged at 22-25° C. for 60 min. The solution was cooled to 7° C. 17.9 g of 50 wt % aqueous NaOH (0.22 mol) and 25.3 g of chloroacetyl chloride (0.22 mol) were added simultaneously over 130 min at a temperature below 16° C. When the reaction was complete, the mixture was vacuum distilled to remove water and ethanol at 27-30° C. and under 26˜27 in Hg vacuum. During the distillation, 720 mL of acetonitrile was added slowly to maintain constant volume (approximately 500 mL). The slurry was filtered to remove sodium chloride. The cake was rinsed with about 100 mL of acetonitrile. Removal of the solvent afforded bis-hydrazide 1-1 (43.2 g, 96.5% yield, 94.4 area % pure by HPLC assay).

¹H-NMR (400 MHz, DMSO-d₆): δ 4.2 (s, 2H), 10.7 (s, 1H), and 11.6 (s, 1H) ppm. ¹³C-NMR (100 MHz, DMSO-d₆): δ 41.0, 116.1 (q, J=362 Hz), 155.8 (q, J=50 Hz), and 165.4 ppm.

Step B: Preparation of 5-(trifluoromethyl)-2-(chloromethyl)-1,3,4-oxadiazole (1-2)

Bishydrazide 1-1 from Step A (43.2 g, 0.21 mol) in ACN (82 mL) was cooled to 5° C. Phosphorus oxychloride (32.2 g, 0.21 mol) was added, maintaining the temperature below 10° C. The mixture was heated to 80° C. and aged at this temperature for 24 h until HPLC showed less than 2 area % of 1-1. In a separate vessel, 260 mL of IPAc and 250 mL of water were mixed and cooled to 0° C. The reaction slurry was charged to the quench keeping the internal temperature below 10° C. After the addition, the mixture was agitated vigorously for 30 min, the temperature was increased to room temperature and the aqueous layer was cut. The organic layer was then washed with 215 mL of water, 215 mL of 5 wt % aqueous sodium bicarbonate and finally 215 mL of 20 wt % aqueous brine solution. HPLC assay yield after work up was 86-92%. Volatiles were removed by distillation at 75-80 mm Hg, 55° C. to afford an oil which could be used directly in Step C without further purification. Otherwise the product can be purified by distillation to afford 1-2 in 70-80% yield.

¹H-NMR (400 MHz, CDCl₃): δ 4.8 (s, 2H) ppm. ¹³C-NMR (100 MHz, CDCl₃): δ32.1, 115.8 (q, J=337 Hz), 156.2 (q, J=50 Hz), and 164.4 ppm.

Step C: Preparation of N-[(2Z)-piperazin-2-ylidene]trifluoroacetohydrazide (1-3)

To a solution of ethylenediamine (33.1 g, 0.55 mol) in methanol (150 mL) cooled at −20° C. was added distilled oxadiazole 1-2 from Step B (29.8 g, 0.16 mol) while keeping the internal temperature at −20° C. After the addition was complete, the resulting slurry was aged at −20° C. for 1 h. Ethanol (225 mL) was then charged and the slurry slowly warmed to −5° C. After 60 min at −5° C., the slurry was filtered and washed with ethanol (60 mL) at −5° C. Amidine 1-3 was obtained as a white solid in 72% yield (24.4 g, 99.5 area wt % pure by HPLC).

¹H-NMR (400 MHz, DMSO-d₆): δ2.9 (t, 2H), 3.2 (t, 2H), 3.6 (s, 2H), and 8.3 (b, 1H) ppm. ¹³C-NMR (100 MHz, DMSO-d₆): δ40.8, 42.0, 43.3, 119.3 (q, J=350 Hz), 154.2, and 156.2 (q, J=38 Hz) ppm.

Step D: Preparation of 3-(trifluoromethyl)-5,6,7,8-tetrahydro[1,2,4]triazolo[4.3-α]pyrazine hydrochloride (1-4)

A suspension of amidine 1-3 (27.3 g, 0.13 mol) in 110 mL of methanol was warmed to 55° C. 37% Hydrochloric acid (11.2 mL, 0.14 mol) was added over 15 min at this temperature. During the addition, all solids dissolved resulting in a clear solution. The reaction was aged for 30 min. The solution was cooled down to 20° C. and aged at this temperature until a seed bed formed (10 min to 1 h). 300 mL of MTBE was charged at 20° C. over 1 h. The resulting slurry was cooled to 2° C., aged for 30 min and filtered. Solids were washed with 50 mL of ethanol:MTBE (1:3) and dried under vacuum at 45° C. Yield of triazole 1-4 was 26.7 g (99.5 area wt % pure by HPLC).

¹H-NMR (400 MHz, DMSO-d₆): δ 3.6 (t, 2H), 4.4 (t, 2H), 4.6 (s, 2H), and 10.6 (b, 2H) ppm; ¹³C-NMR (100 MHz, DMSO-d₆): δ: 39.4, 39.6, 41.0, 118.6 (q, J=325 Hz), 142.9 (q, J=50 Hz), and 148.8 ppm. 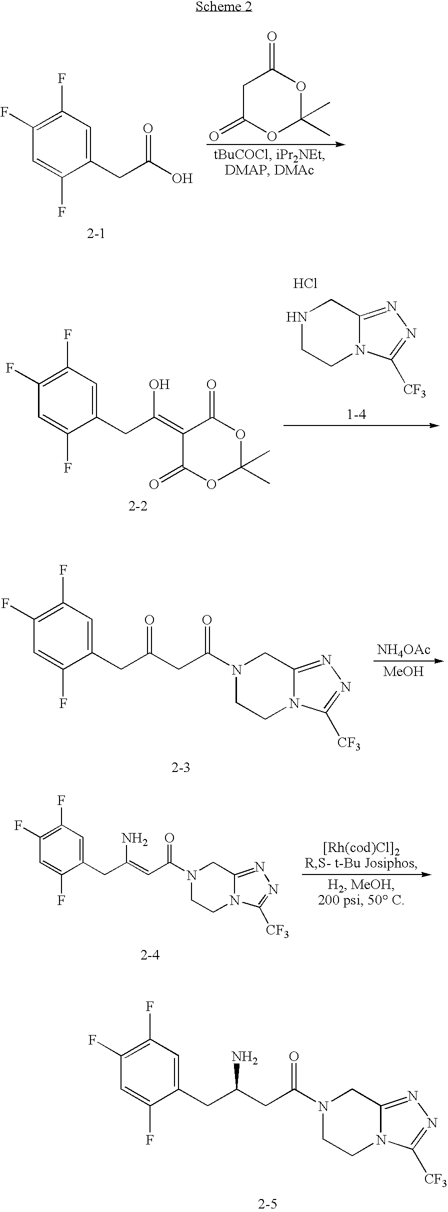
Step A: Preparation of 4-oxo4-[3-(trifluoromethvl)-5,6-dihydro[1,2,4]triazolo[4,3-α]pyrazin-7(8H)-yl]-1-(2,4,5-trifluorophenl)butan-2-one (2-3)

2,4,5-Trifluorophenylacetic acid (2-1) (150 g, 0.789 mol), Meldrum's acid (125 g, 0.868 mol), and 4-(dimethylamino)pyridine (DMAP) (7.7 g, 0063 mol) were charged into a 5 L three-neck flask. N,N-Dimethylacetamide (DMAc) (525 mL) was added in one portion at room temperature to dissolve the solids. N,N-diisopropylethylamine (282 mL, 1.62 mol) was added in one portion at room temperature while maintaining the temperature below 40° C. Pivaloyl chloride (107 mL, 0.868 mol) was added dropwise over 1 to 2 h while maintaining the temperature between 0 and 5° C. The reaction mixture was aged at 5° C. for 1 h. Triazole hydrochloride 1-4 (180 g, 0.789 mol) was added in one portion at 40-50° C. The reaction solution was aged at 70° C. for several h. 5% Aqueous sodium hydrogencarbonate solution (625 mL) was then added dropwise at 20-45° C. The batch was seeded and aged at 20-30° C. for 1-2 h. Then an additional 525 mL of 5% aqueous sodium hydrogencarbonate solution was added dropwise over 2-3 h. After aging several h at room temperature, the slurry was cooled to 0-5° C. and aged 1 h before filtering the solid. The wet cake was displacement-washed with 20% aqueous DMAc (300 mL), followed by an additional two batches of 20% aqueous DMAc (400 mL), and finally water (400 mL). The cake was suction dried at room temperature. The isolated yield of final product 2-3 was 89%.

Step B: Preparation of (2Z)-4-oxo-4-[3-(trifluoromethy )-5,6-dihydro [1,2,4]triazolo[4,3-α]pyrazin-7(8H)-yl]-1-(2,4,5-trifluorophenyl)but-2-en-2-amine(2-4)

A 5 L round-bottom flask was charged with methanol (100 mL), the ketoamide 2-3 (200 g), and ammonium acetate (110.4 g). Methanol (180 mL) and 28% aqueous ammonium hydroxide (58.6 mL) were then added keeping the temperature below 30° C. during the addition. Additional methanol (100 mL) was added to the reaction mixture. The mixture was heated at reflux temperature and aged for 2 h. The reaction was cooled to room temperature and then to about 5° C. in an ice-bath. After 30 min, the solid was filtered and dried to afford 2-4 as a solid (180 g); m.p. 271.2° C.

Step C: Preparation of (2R)-4-oxo-4-[3-(trifluoromethl)-5,6-dihydro [1,2,4]triazolo[4,3-α]pyrazin-7(8H)-yl]-1-(2,4,5-trifluorophenyl)butan-2-amine (2-5)

Into a 500 ml flask were charged chloro(1,5-cyclooctadiene)rhodium(I) dimer {[Rh(cod)C]₂}(292 mg, 1.18 mmol) and (R,S) t-butyl Josiphos (708 mg, 1.3 mmol) under a nitrogen atmosphere. Degassed MeOH was then added (200 mL) and the mixture was stirred at room temperature for 1 h. Into a 4 L hydrogenator was charged the enamine amide 2-4 (118 g, 0.29 mol) along with MeOH (1 L). The slurry was degassed. The catalyst solution was then transferred to the hydrogenator under nitrogen. After degassing three times, the enamine amide was hydrogenated under 200 psi hydrogen gas at 50° C. for 13 h. Assay yield was determined by HPLC to be 93% and optical purity to be 94% ee.

The optical purity was farther enhanced in the following manner. The methanol solution from the hydrogenation reaction (18 g in 180 mL MeOH) was concentrated and switched to methyl t-butyl ether (MTBE) (45 mL). Into this solution was added aqueous H₃PO₄ solution (0.5 M, 95 mL). After separation of the layers, 3N NaOH (35 mL) was added to the water layer, which was then extracted with MOBE (180 mL+100 mL). The MTBE solution was concentrated and solvent switched to hot toluene (180 mL, about 75° C.). The hot toluene solution was then allowed to cool to 0° C. slowly (5-10 h). The crystals were isolated by filtration (13 g, yield 72%, 98-99% ee); m.p. 114.1 - 115.7° C.

¹H NMR (300 MHz, CD₃CN): δ 7.26 (m), 7.08 (m), 4.90 (s), 4.89 (s), 4.14 (m), 3.95 (m), 3.40 (m), 2.68 (m), 2.49 (m), 1.40 (bs).

Compound 2-5 exists as amide bond rotamers. Unless indicated, the major and minor rotamers are grouped together since the carbon-13 signals are not well resolved:

¹³C NMR (CD₃CN): δ 171.8, 157.4 (ddd, J_{CF}=242.4, 9.2, 2.5 Hz), 152.2 (major), 151.8 (minor), 149.3 (ddd; J_{CF}=246.7, 14.2, 12.9 Hz), 147.4 (ddd, J_{CF}=241.2, 12.3, 3.7 Hz), 144.2 (q, J_{CF}=38.8 Hz), 124.6 (ddd, J_{CF}=18.5, 5.9,4.0 Hz), 120.4 (dd, J_{CF}=19.1, 6.2 Hz), 119.8 (q, J_{CF}=268.9 Hz), 106.2 (dd, J_{CF}=29.5, 20.9 Hz), 50.1, 44.8, 44.3 (minor), 43.2 (minor), 42.4, 41.6 (minor), 41.4, 39.6, 38.5 (minor), 36.9.

The crystalline free base 2-5 can also be isolated as follows:
(a) The reaction mixture upon completion of the hydrogenation step is charged with 25 wt % of Ecosorb C-941. The mixture is stirred under nitrogen for one h and then filtered. The cake is washed with 2 L/kg of methanol. Recovery of free base is about 95% and optical purity about 95% ee.
(b) The freebase solution in methanol is concentrated to 3.5-4.0 L/kg volume (based on free base charge) and then solvent-switched into isopropanol (IPA) to final volume of 3.0 L/kg IPA.
(c) The slurry is heated to 40° C. and aged 1 h at 40° C. and then cooled to 25° C. over 2 h.
(d) Heptane (7 L/kg) is charged over 7 h and the slurry stirred for 12 h at 22-25° C. The supernatant concentration before filtering is 10-12 mg/g.
(e) The slurry is filtered and the solid washed with 30% PA/heptane (2 L/kg).
(f) The solid is dried in a vacuum oven at 40° C.
(g) The optical purity of the free base is about 99% ee.

The following high-performance liquid chromatographic (HPLC) conditions were used to determine percent conversion to product:
Column: Waters Symmetry C18, 250 mm×4.6 mm
Eluent: Solvent A: 0.1 vol % HClO₄/H₂O
   Solvent B: acetonitrile
Gradient: 0 min 75% A: 25% B
   10 min 25% A:75% B
   12.5 min 25% A: 75% B
   15 min 75% A: 25% B
Flow rate: 1 mL/min
Injection Vol.: 10 μL
UV detection: 210 nm
Column temp.: 40° C.
Retention times: compound 2-4: 9.1 min
   compound 2-5: 5.4 min
   tBu Josiphos: 8.7 min

The following high-perfornance liquid chromatographic (HPLC) conditions were used to determine optical purity:
Column: Chirapak, AD-H, 250 mm×4.6 mm
Eluent: Solvent A: 0.2 vol. % diethylamine in heptane
   Solvent B: 0.1 vol % diethylamine in ethanol
Isochratic Run Time: 18 min
Flow rate: 0.7 mL/min
Injection Vol.: 7 μL
UV detection: 268 nm
Column temp.: 35° C.
Retention times: (R)-amine 2-5: 13.8 min
   (S)-amine 2-5: 11.2 min
      Preparation of (2R)4-oxo-4-[3-(trifluoromethyl)-5,6-dihydro[1,2,4]triazolo[4,3-α]pyrazin-7(8H)-yl]-1-(2,4,5-trifluorophenyl)butan-2-amine dihydrogenphosphate anhydrate Form I and III mixture

A 250 mL round bottom flask equipped with an overhead stirrer, heating mantle and thermocouple, was charged with 60 mL of ethanol, 19 mL water, 15.0 g (36.9 mmol) of (2R)-4-oxo-4-[3-(trifluoromethyl)-5,6-dihydro[1,2,4]triazolo[4,3-α]pyrazin-7(8H)-yl]-1-(2,4,5-trifluorophenyl)butan-2-amine freebase, and 4.25 g (36.9 mmol) of 85% aqueous phosphoric acid. The mixture was heated to 75 to 78° C. A thick white precipitate formed at lower temperatures but dissolved upon reaching 75° C. The solution was cooled to 68° C. and then held at that temperature for 4-8 h. A slurry bed of solids of ethanol solvate formed during this age time. The slurry was then cooled at a rate of 4° C./h to 21° C. and then held overnight. 70 mL of ethanol was then added to the slurry of ethanol solvate. After 1 h the slurry of ethanol solvate was filtered and washed with 45 mL ethanol. The solids were dried in a vacuum oven at 40° C. for 18 h. 17.1 g of solids that were a mixture of Form I and Form III were recovered. The solids were found to greater than 99.8% pure by HPLC area percentage (HPLC conditions same as those 25 given above). The crystal form of the solids was shown to be a mixture of anhydrate Forms I and III by X-ray powder diffraction and solid state NMR spectroscopy, with Form I predominating.

### EXAMPLE 2

(2R)4-Oxo4-[3-(trifluoromethyl)-5,6-dihydro[1,2,4]triazolo[4,3-α]pyrazin-7(8H)-yl]-1-(2,4,5-trifluorophenyl)butan-2-amine freebase 2-5 in isoamyl alcohol solution (˜200 mg/g) was added to the crystallizer. A seed was then added, followed by isoamyl alcohol and water to constitute a 96% isoamyl alcohol and 4% water mixture. The mixture was first aged, and then heated up to about 50° C. About 1 equivalent of phosphoric acid in 96% isoamyl alcohol and 4% water (to achieve a final batch concentration of 85 mg/g) was then added to the slurry to crystallize the anhydrate Form I. The slurry as aged and then cooled to room temperature. The solids were filtered and washed with isoamyl lcohol. The wet solids were dried at 75-80° C. The crystal form of the solids was shown to be a ixture of anhydrate Forms I and III by X-ray powder diffraction and solid state NMR spectroscopy, with Form I predominating.

X-ray powder diffraction studies are widely used to characterize molecular structures, crystallinity, and polymorphism. The X-ray powder diffraction patterns of the crystalline polymorphs of the present invention were generated on a Philips Analytical X'Pert PRO X-ray Diffraction System with PW3040/60 console. A PW3373/00 ceramic Cu LEF X-ray tube K-Alpha radiation was used as the source.

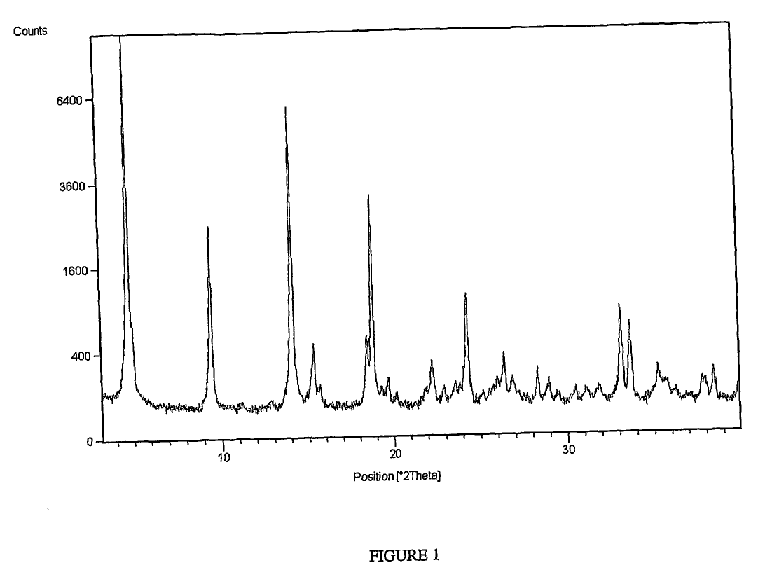

FIG. 1 shows the X-ray diffraction pattern for the crystalline anhydrate Form I. The anhydrate Form I exhibited characteristic reflections corresponding to d-spacings of 18.42, 9.35, and 6.26 angstroms. The anhydrate Form I was further characterized by reflections corresponding to d-spacings of 5.78, 4.71, and 3.67 angstroms. The anhydrate Form I was even further characterized by reflections corresponding to d-spacings of 3.99, 2.71, and 2.66 angstroms.

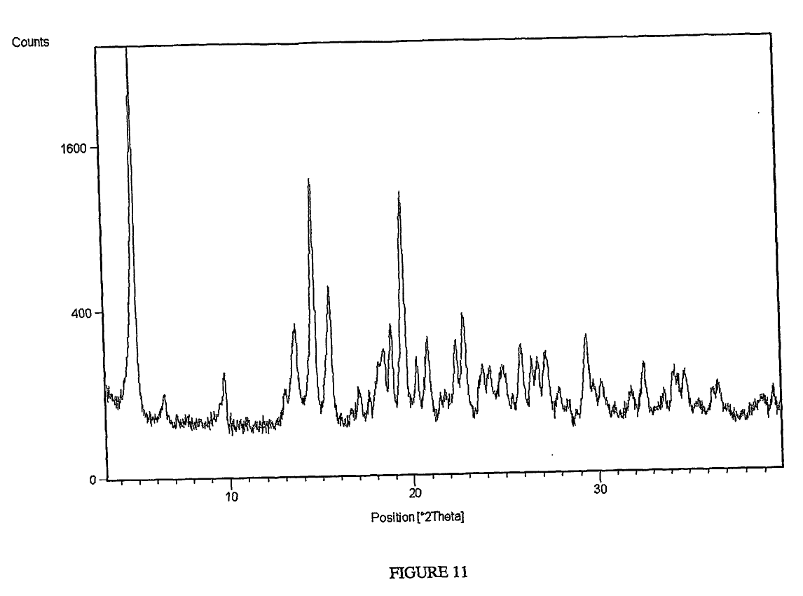

FIG. 11 shows the X-ray diffraction pattern for the crystalline anhydrate Form III. The anhydrate Form III exhibited characteristic reflections corresponding to d-spacings of 17.88, 6.06, and 4.26 angstroms. The anhydrate Form III was further characterized by reflections corresponding to d-spacings of 9.06, 5.71, and 4.55 angstroms. The anhydrate Form m was even further characterized by reflections corresponding to d-spacings of 13.69, 6.50, and 3.04 angstroms.

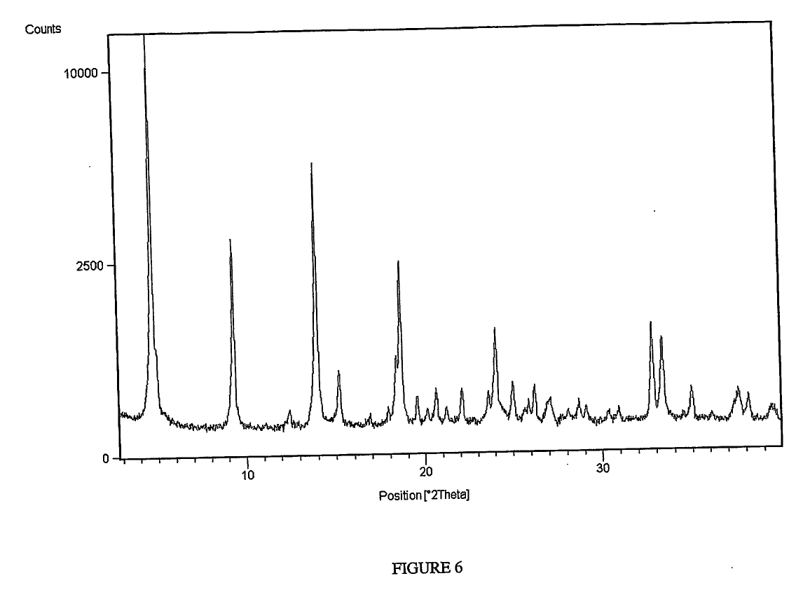

FIG. 6 shows the X-ray diffraction pattern for the crystalline desolvated anhydrate Form II. The desolvated anhydrate Form II exhibited characteristic reflections corresponding to d-spacings of 7.09, 5.27, and 4.30 angstroms. The desolvated anhydrate Form II was further characterized by reflections corresponding to d-spacings of 18.56, 9.43 and 4.19 angstroms. The desolvated anhydrate Form II was even further characterized by reflections corresponding to d-spacings of 6.32, 5.82, and 3.69 angstroms.

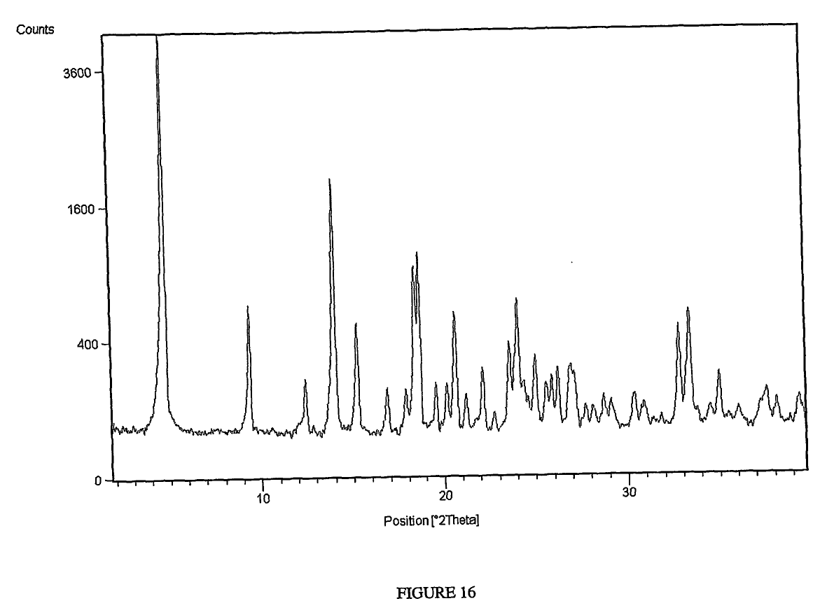

FIG. 16 shows the X-ray diffraction pattern for the crystalline ethanol solvate. The crystalline ethanol solvate exhibited the same XRPD pattern as desolvated anhydrate Form II with characteristic reflections corresponding to d-spacings of 7.09, 5.27, and 4.30 angstroms. The crystalline ethanol solvate was further characterized by reflections corresponding to d-spacings of 18.56, 9.43 and 4.19 angstroms. The crystalline ethanol solvate was even further characterized by reflections corresponding to d-spacings of 6.32, 5.82, and 3.69 angstroms.

In addition to the X-ray powder diffraction patterns described above, the crystalline polymorphic forms of Compound I of the present invention were further characterized by their solid-state carbon-13 and fluorine-19 nuclear magnetic resonance (NMR) spectra. The solid-state carbon-13 NMR spectrum was obtained on a Bruker DSX 40OWB NMR system using a Bruker 4 mm double resonance CPMAS probe. The carbon-13 NMR spectrum utilized proton/carbon-13 cross-polarization magic-angle spinning with variable-amplitude cross polarization. The sample was spun at 15.0 kHz, and a total of 1024 scans were collected with a recycle delay of 5 seconds. A line broadening of 40 Hz was applied to the spectrum before FT was performed. Chemical shifts are reported on the TMS scale using the carbonyl carbon of glycine (176.03 p.p.m.) as a secondary reference.

The solid-state fluorine-19 NMR spectrum was obtained on a Bruker DSX 400WB NMR system using a Bruker 4 mm CRAMPS probe. The NMR spectrum utilized a simple pulse-acquire pulse program. The samples were spun at 15.0 kHz, and a total of 128 scans were collected with a recycle delay of 5 seconds. A vespel endcap was utilized to minimize fluorine background. A line broadening of 100 Hz was applied to the spectrum before FT was performed. Chemical shifts are reported using poly(tetrafluoroethylene) (teflon) as an external secondary reference which was assigned a chemical shift of −122 ppm

DSC data were acquired using TA Instruments DSC 2910 or equivalent instrumentation. Between 2 and 6 mg of sample were weighed into an open pan. This pan was then crimped and placed at the sample position in the calorimeter cell. An empty pan was placed at the reference position. The calorimeter cell was closed and a flow of nitrogen was passed through the cell. The heating program was set to heat the sample at a heating rate of 10° C./min to a temperature of approximately 250° C. The heating program was started. When the run was completed, the data were analyzed using the DSC analysis program contained in the system software. The melting endotherm was integrated between baseline temperature points that are above and below the temperature range over which the endotherm was observed. The data reported are the onset temperature, peak temperature and enthalpy.

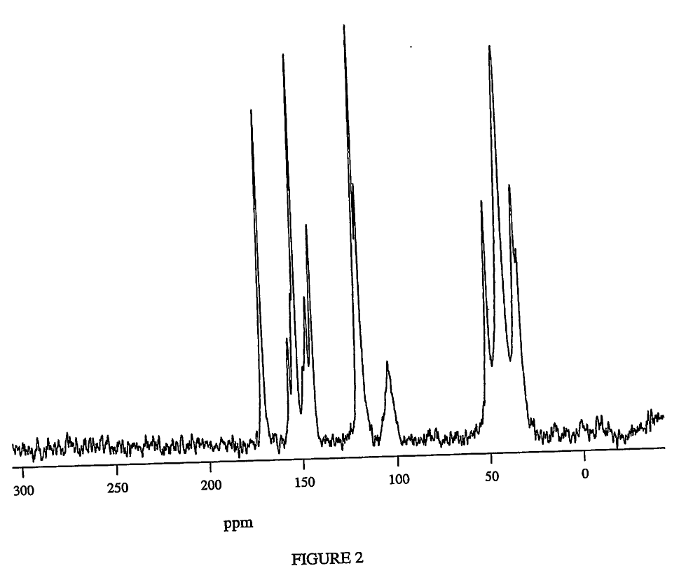

FIG. 2 shows the solid-state carbon-13 CPMAS NMR spectrum for the crystalline anhydrate Form I of Compound I.

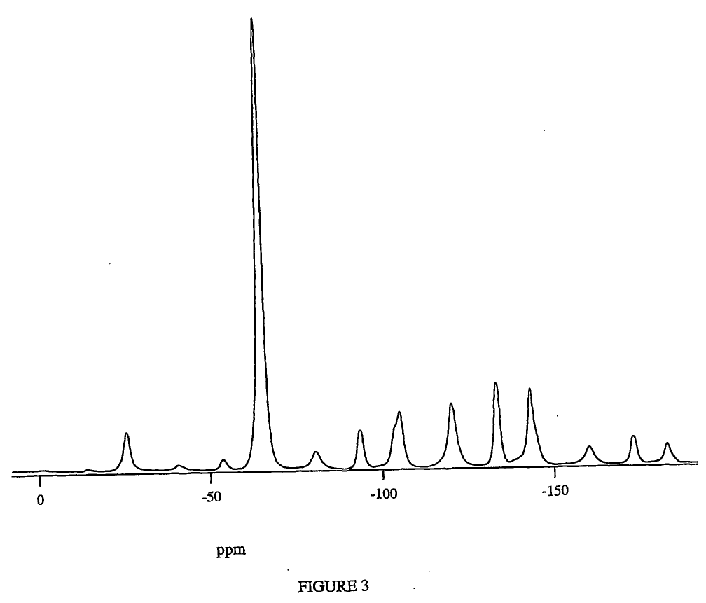

FIG. 3 shows the solid-state fluorine-19 MAS NMR spectrum for the crystalline anhydrate Form I of Compound I. Form I exhibited characteristic signals with chemical shift values of −65.3, −105.1, and −120.4 p.p.m Further characteristic of Form I are the signals with chemical shift values of −80.6, −93.5, and −133.3 p.p.m.

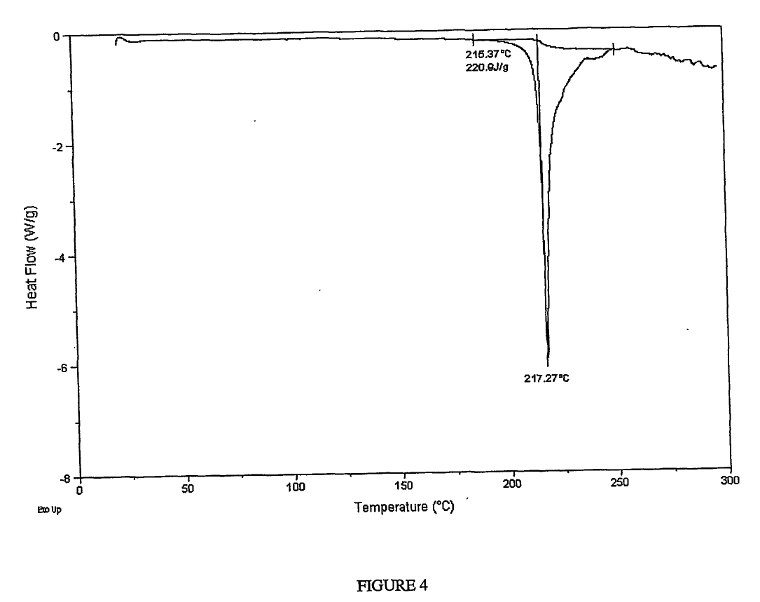

FIG. 4 shows the differential calorimetry scan for the crystalline anhydrate Form I. Form I exhibited a melting endotherm with an onset temperature of 215° C., a peak temperature of 217° C., and an enthalpy of 221J/g.

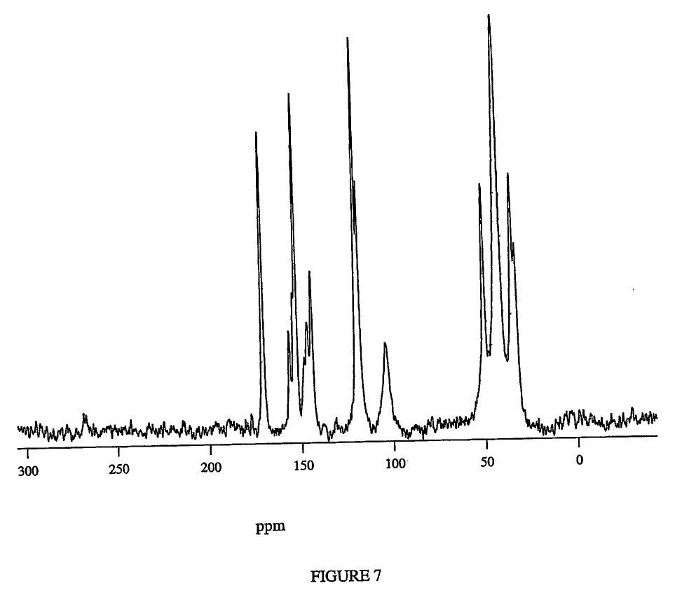

FIG. 7 shows the solid-state carbon-13 CPMAS NMR spectrum for the crystalline desolvated anhydrate Form II of Compound I.

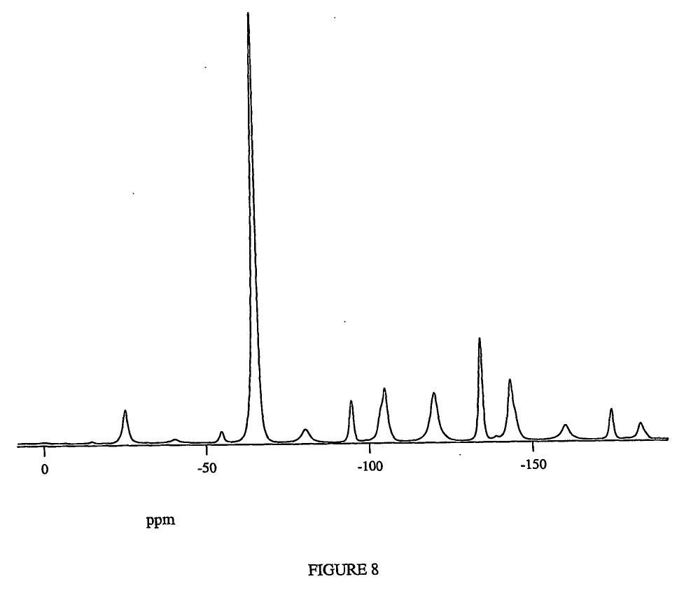

FIG. 8 shows the solid-state fluorine-19 MAS NMR spectrum for the crystalline desolvated anhydrate Form II of Compound I. Form II exhibited characteristic signals with chemical shift values of −65.1, −104.9, and −120.1 p.p.m. Further characteristic of Form II are the signals with chemical shift values of −80.3, −94.5, −134.4, and −143.3 p.p.m.

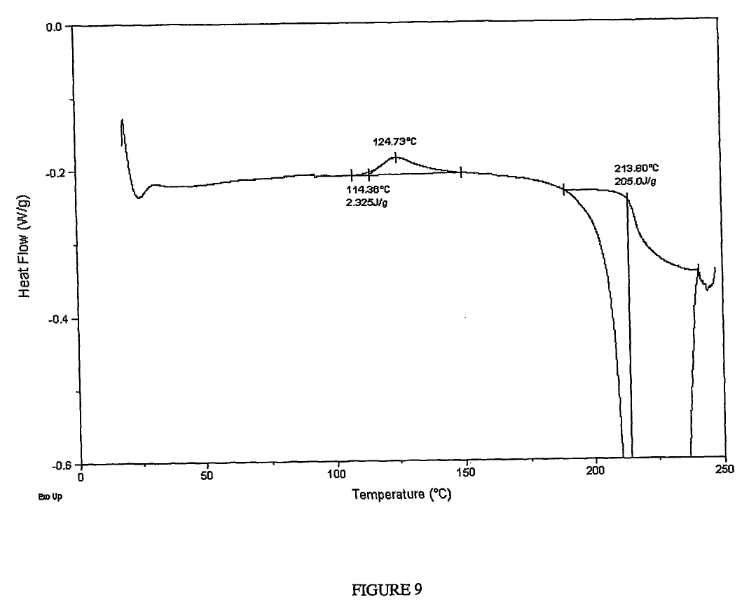

FIG. 9 shows the differential calorimetry scan for crystalline desolvated anhydrate Form II. Form II exhibited a solid-solid transition exotherm to crystalline anhydrate Form I with an onset temperature of 114° C., a peak temperature of 125° C., and an enthalpy of 2.3 J/g.

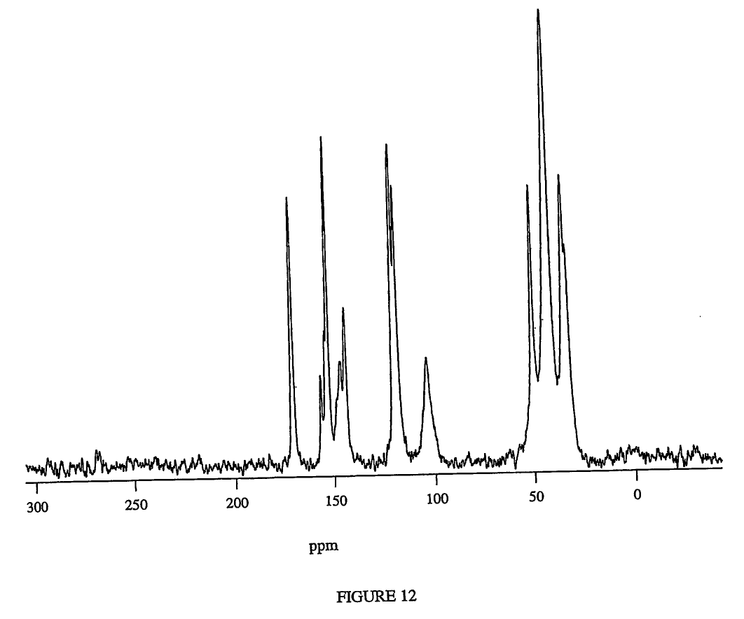

FIG. 12 shows the solid-state carbon-13 CPMAS NMR spectrum for the crystalline anhydrate Form III of Compound I.

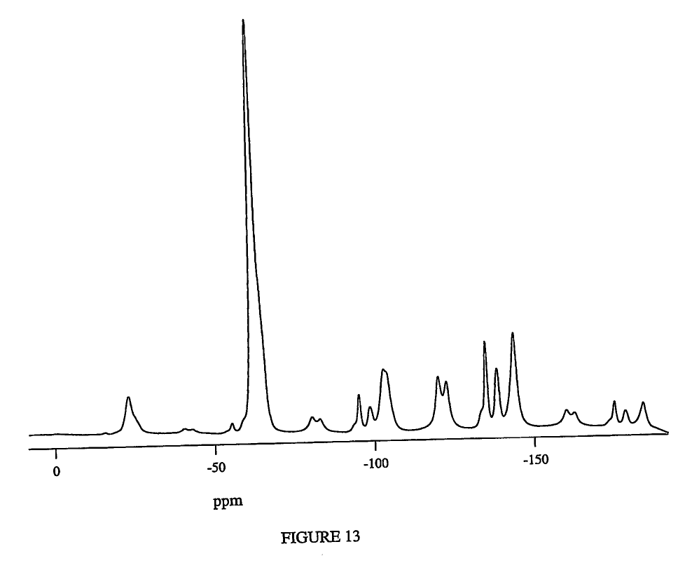

FIG. 13 shows the solid-state fluorine-19 MAS NMR spectrum for the crystalline anhydrate Form III of Compound I. Form III exhibited characteristic signals with chemical shift values of −63.0, −103.1, and −120.2 p.p.m Further characteristic of Form III are the signals with chemical shift values of −95.3, −98.7, −135.2, and −144.0 p.p.m

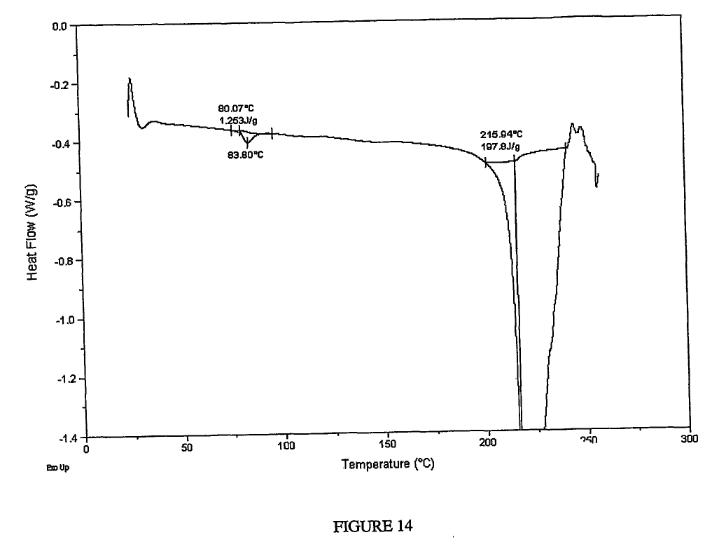

FIG. 14 shows the differential calorimetry scan for crystalline anhydrate Form III. Form III exhibited a solid-solid transition endotherm to crystalline anhydrate Form I with an onset temperature of 80° C., a peak temperature of 84° C., and an enthalpy of 1.3 J/g.

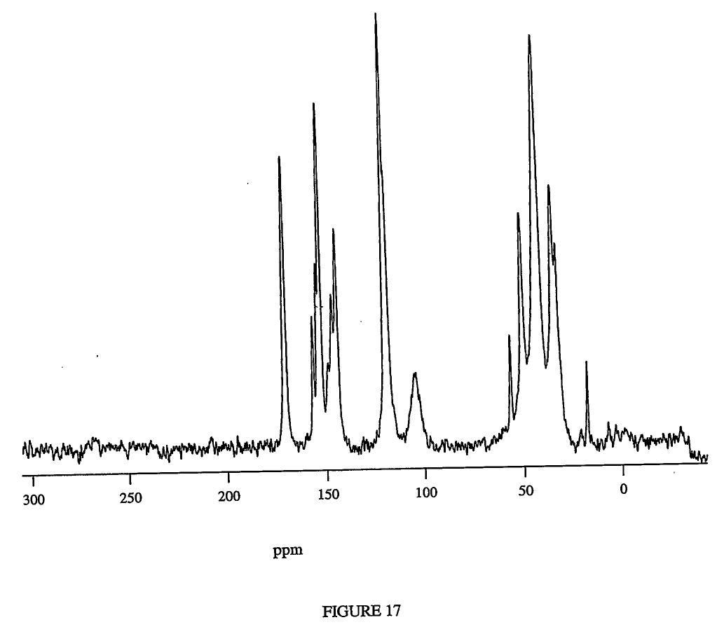

FIG. 17 shows the solid-state carbon-13 CPMAS NMR spectrum for the crystalline ethanol solvate of Compound I.

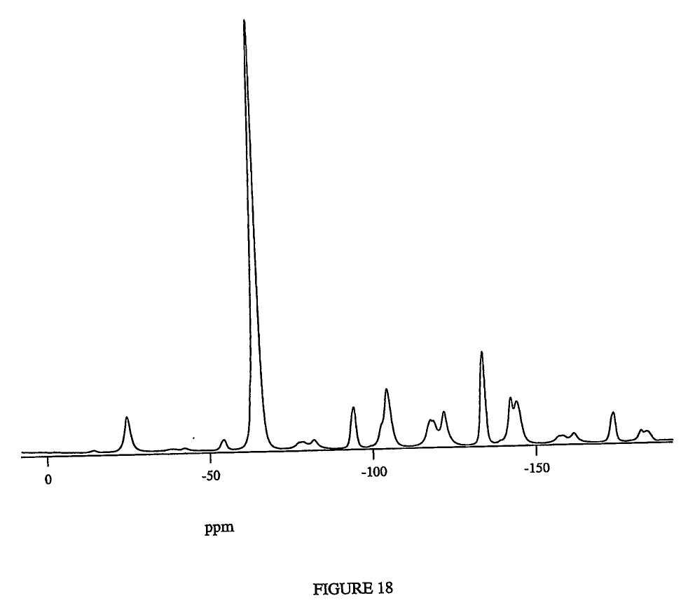

FIG. 18 shows the solid-state fluorine-19 MAS NMR spectrum for the crystalline ethanol solvate of Compound I. The ethanol solvate exhibited characteristic signals with chemical shift values of −64.7, −104.5, and −121.9 p.p.m. Further characteristic of ethanol solvate are the signals with chemical shift values of −94.3, −117.7, −131.2, and −142.6 p.p.m.

The crystalline Compound I anhydrate Form I or Form III or mixture thereof of the present invention has a phase purity of at least about 5% of Form I or Form III or mixture thereof with the above X-ray powder diffraction, fluorine-19 MAS NMR, carbon-13 CPMAS NMR, and DSC physical characteristics. In one embodiment the phase purity is at least about 10% of Form I or Form III or mixture thereof with the above solid-state physical characteristics. In a second embodiment the phase purity is at least about 25% of Form I or Form III or mixture thereof with the above solid-state physical characteristics. In a third embodiment the phase purity is at least about 50% of Form I or Form III or mixture thereof with the above solid-state physical characteristics. In a fourth embodiment the phase purity is at least about 75% of Form I or Form III or mixture thereof with the above solid-state physical characteristics. In a fifth embodiment the phase purity is at least about 90% of Form I or Form III or mixture thereof with the above solid-state physical characteristics. In a sixth embodiment the crystalline Compound I is the substantially phase pure Form I or Form III or mixture thereof with the above solid-state physical characteristics. By the term “phase purity” is meant the solid state purity of the Compound anhydrate Form I or Form III or mixture thereof with regard to another particular crystalline or amorphous form of Compound I as determined by the solid-state physical methods described in the present application.

Examples of Pharmaceutical Compositions:

1) Direct compression process:

Compound I anhydrate Form I or Form III or a mixture thereof (API) was formulated into a tablet by a direct compression process. A 100 mg potency tablet is composed of 124 mg of the API, 130 mg microcrystalline cellulose, 130 mg of mannitol (or 130 mg of dicalcium phosphate), 8 mg of croscarmellose sodium, 8 mg of magnesium stearate and 16 mg of Opadry white (proprietary coating material made by Colorcon, West Point, Pa.). The API, microcrystalline cellulose, mannitol (or dicalcium phosphate), and croscarmellose sodium were first blended, and the mixture was then lubricated with magnesium stearate and pressed into tablets. The tablets were then film coated with Opadry White.

2) Roller compaction process:

Compound I anhydrate Form I or Form III or a mixture thereof was formulated into a tablet by a roller compaction process. A 100 mg potency tablet is composed of 124 mg of the API, 195 mg microcrystalline cellulose, 65 mg of mannitol, 8 mg of croscarmellose sodium, 8 mg of magnesium stearate and 16 mg of Opadry white (proprietary coating material made by Colorcon, West Point, Pa.). The API, microcrystalline cellulose, mannitol, and croscarmellose sodium were first blended, and the mixture was then lubricated with one third the total amount of magnesium stearate and roller compacted into ribbons. These ribbons were then milled and the resulting granules were lubricated with the remaining amount of the magnesium stearate and pressed into tablets. The tablets were then film coated with Opadry White.

3) An intravenous (i.v.) aqueous formulation is defined as the anhydrate Form I or Form III or a mixture thereof of Compound I in 10 mM sodium acetate/0.8% saline solution at pH 4.5±0.2. For a formulation with a concentration of 4.0 mg/mL, 800 mg of NaCl is dissolved in 80 mL of water, then 57.5 μL of glacial acetic acid is added, followed by 496 mg of the anhydrate Form I or Form III or a mixture thereof. The pH is adjusted to 4.5±0.2 with 0.1 N NaOH solution. The final volume is adjusted to 100 mL with water. A 2.0-mg/mL solution can be made by dilution of 50.0 mL of the 4.0-mg/mL solution to 100.0 mL with placebo. A 1.0-mg/mL solution can be made by dilution of 25.0 mL of the 4.0-mg/mL solution to 100.0 mL with placebo.

1. A dihydrogenphosphate salt of (2R)-4-oxo-4-[3-(trifluoromethyl)-5,6-dihydro[1,2,4]triazolo[4,3-α]pyrazin-7(8H)-yl]-1-(2,4,5-trifluorophenyl)butan-2-amine of structural formula I: 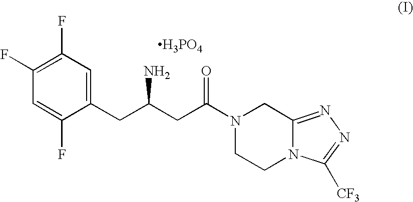
characterized as being a crystalline anhydrate Form I.

2. The crystalline anhydrate Form I of claim 1 characterized by characteristic reflections obtained from the X-ray powder diffraction pattern at spectral d-spacings of 18.42, 9.35, and 6.26 angstroms.

3. The crystalline anhydrate Form I of claim 2 further characterized by characteristic reflections obtained from the X-ray powder diffraction pattern at spectral d-spacings of 5.78, 4.71, and 3.67 angstroms.

4. The crystalline anhydrate Form I of claim 3 further characterized by characteristic reflections obtained from the X-ray powder diffraction pattern at spectral d-spacings of 3.99, 2.71, and 2.66 angstroms.

5. The crystalline anhydrate Form I of claim 4 further characterized by the X-ray powder diffraction pattern of FIG. 1.

6. The crystalline anhydrate Form I of claim 1 characterized by a solid-state fluorine-19 MAS nuclear magnetic resonance spectrum showing signals at −65.3,−105.1, and −120.4 p.p.m.

7. The crystalline anhydrate Form I of claim 6 further characterized by a solid-state fluorine-19 MAS nuclear magnetic resonance spectrum showing signals at −80.6, −93.5, and −133.3 p.p.m.

8. The crystalline anhydrate Form I of claim 7 further characterized by the solid-state fluorine-19 MAS nuclear magnetic resonance spectrum of FIG. 3.

9. The crystalline anhydrate Form I of claim 1 characterized by the solid-state carbon-13 CPMAS nuclear magnetic resonance spectrum of FIG. 2.

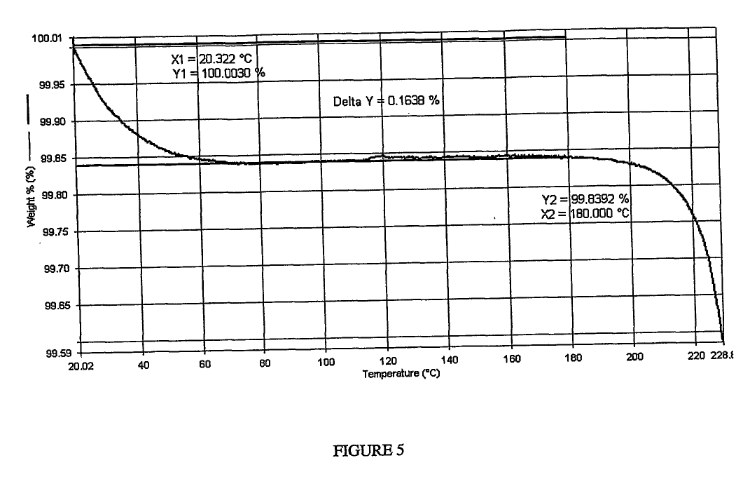

10. The crystalline anhydrate Form I of claim 1 characterized by the thermogravimetric analysis curve of FIG. 5.

11. The crystalline anhydrate Form I of claim 1 characterized by the differential scanning calorimetric (DSC) curve of FIG. 4.

12. A dihydrogenphosphate salt of (2R)-4-oxo-4-[3-(trifluoromethyl)-5,6-dihydro[1,2,4]triazolo[4,3-α]pyrazin-7(8H)-yl]-1-(2,4,5-trifluorophenyl)butan-2-amine of structural formula I: 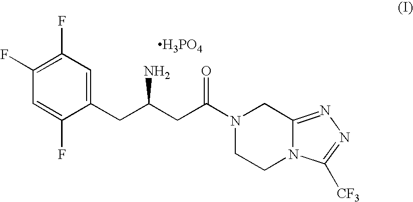
characterized as being a crystalline anhydrate Form III.

13. The crystalline anhydrate Form III of claim 12 characterized by characteristic reflections obtained from the X-ray powder diffraction pattern at spectral d-spacings of 17.88, 6.06, and 4.26 angstroms.

14. The crystalline anhydrate Form imi of claim 13 further characterized by characteristic reflections obtained from the X-ray powder diffraction pattern at spectral d-spacings of 9.06, 5.71, and 4.55 angstroms.

15. The crystalline anhydrate Form III of claim 14 further characterized by characteristic reflections obtained from the X-ray powder diffraction pattern at spectral d-spacings of 13.69, 6.50, and 3.04 angstroms.

16. The crystalline anhydrate Form III of claim 15 further characterized by the X-ray powder diffraction pattern of FIG. 11.

17. The crystalline anhydrate Form III of claim 12 characterized by a solid-state fluorine-19 MAS nuclear magnetic resonance spectrum showing signals at −63.0, −103.1, and −120.2 p.p.m.

18. The crystalline anhydrate Form III of claim 17 further characterized by a solid-state fluorine-19 MAS nuclear magnetic resonance spectrum showing signals at −95.3, −98.7, −135.2, and −144.0 p.p.m.

19. The crystalline anhydrate Form III of claim 18 further characterized by the solid-state fluorine-19 MAS nuclear magnetic resonance spectrum of FIG. 13.

20. The crystalline anhydrate Form III of claim 12 characterized by the solid-state carbon-13 CPMAS nuclear magnetic resonance spectrum of FIG. 12.

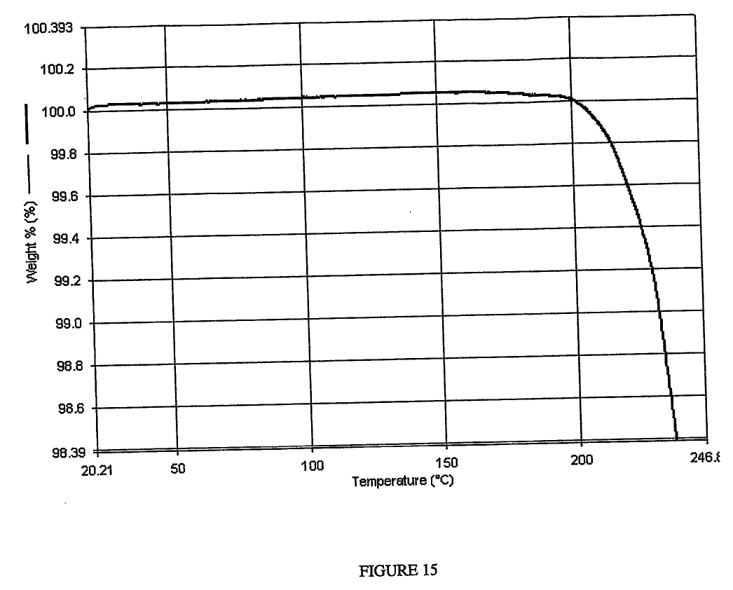

21. The crystalline anhydrate Form III of claim 12 characterized by the thermogravimetric analysis curve of FIG. 15.

22. The crystalline anhydrate Form III of claim 12 characterized by the differential scanning calorimetric (DSC) curve of FIG. 14.

23. A dihydrogenphosphate salt of (2R)-4-oxo-4-[3-(trifluoromethyl)-5,6-dihydro[1,2,4]triazolo[4,3-α]pyrazin-7(8H)-yl]-1-(2,4,5-trifluorophenyl)butan-2-amine of structural formula I: 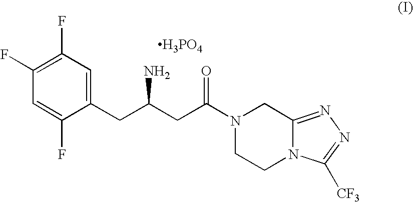
characterized as being a crystalline desolvated anhydrate Form II.

24. The crystalline desolvated anhydrate Form II of claim 23 characterized by characteristic reflections obtained from the X-ray powder diffraction pattern at spectral d-spacings of 7.09, 5.27, and 4.30 angstroms.

25. The crystalline desolvated anhydrate Form II of claim 24 further characterized by characteristic reflections obtained from the X-ray powder diffraction pattern at spectral d-spacings of 18.56, 9.43, and 4.19 angstroms.

26. The crystalline desolvated anhydrate Form II of claim 25 further characterized by characteristic reflections obtained from the X-ray powder diffraction pattern at spectral d-spacings of 6.32, 5.82, and 3.69 angstroms.

27. The crystalline desolvated anhydrate Form II of claim 26 further characterized by the X-ray powder diffraction pattern of FIG. 6.

28. The crystalline desolvated anhydrate Form II of claim 23 characterized by a solid-state fluorine-19 MAS nuclear magnetic resonance spectrum showing signals at −65.1, −104.9, and −120.1 p.p.m.

29. The crystalline desolvated anhydrate Form II of claim 28 further characterized by a solid-state fluorine-19 MAS nuclear magnetic resonance spectrum showing signals at −80.3, −94.5, −134.4, and −143.3 p.p.m.

30. The crystalline desolvated anhydrate Form II of claim 29 further characterized by the solid-state fluorine-19 MAS nuclear magnetic resonance spectrum of FIG. 8.

31. The crystalline desolvated anhydrate Form II of claim 23 characterized by the solid-state carbon-13 CPMAS nuclear magnetic resonance spectrum of FIG. 7.

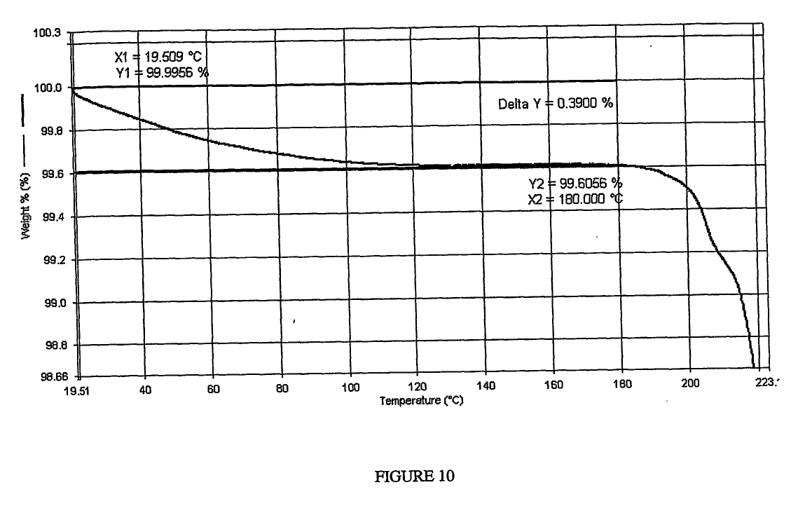

32. The crystalline desolvated anhydrate Form II of claim 23 characterized by the thermogravimetric analysis curve of FIG. 10.

33. The crystalline desolvated anhydrate Form II of claim 23 characterized by the differential scanning calorimetric (DSC) curve of FIG. 9.

34. A dihydrogenphosphate salt of (2R)-4-oxo-4-[3-(trifluoromethyl)-5,6-dihydro[1,2,4]triazolo[4,3-α]pyrazin-7(8H)-yl]-1-(2,4,5-trifluorophenyl)butan-2-amine of structural formula I: 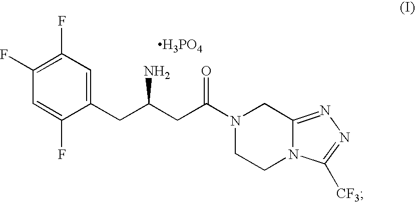
characterized as being a crystalline solvate wherein the solvate is selected from the group consisting of acetone solvate, acetonitrile solvate, methanolate, ethanolate, 1-propanolate, and 2-propanolate.

35. The crystalline solvate of claim 34 wherein said solvate is an ethanolate.

36. The crystalline ethanolate of claim 35 characterized by characteristic reflections obtained from the X-ray powder diffraction pattern at spectral d-spacings of 7.09, 5.27, and 4.30 angstroms.

37. The crystalline ethanolate of claim 36 further characterized by characteristic reflections obtained from the X-ray powder diffraction pattern at spectral d-spacings of 18.56, 9.43, and 4.19 angstroms.

38. The crystalline ethanolate of claim 37 further characterized by characteristic reflections obtained from the X-ray powder diffraction pattern at spectral d-spacings of 6.32, 5.82, and 3.69 angstroms.

39. The crystalline ethanolate of claim 38 further characterized by the X-ray powder diffraction pattern of FIG. 16.

40. The crystalline ethanolate of claim 35 characterized by a solid-state fluorine-19 MAS nuclear magnetic resonance spectrum showing signals at −64.7, −104.5, and −121.9 p.p.m.

41. The crystalline ethanolate of claim 40 further characterized by a solid-state fluorine-19 MAS nuclear magnetic resonance spectrum showing signals at −94.3,−117.7, −131.2, and −142.6 p.p.m.

42. The crystalline ethanolate of claim 41 further characterized by the solid-state fluorine-19 MAS nuclear magnetic resonance spectrum of FIG. 18.

43. The crystalline ethanolate of claim 35 characterized by the solid-state carbon-13 CPMAS nuclear magnetic resonance spectrum of FIG. 17.

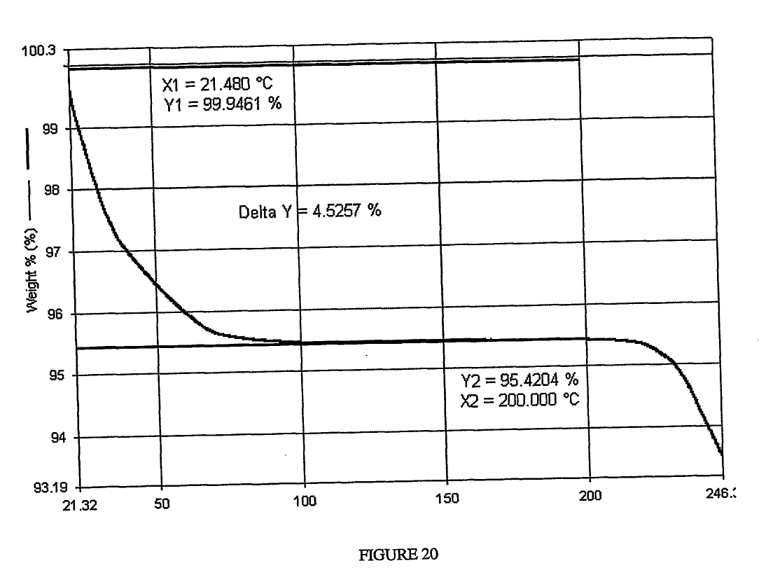

44. The crystalline ethanolate of claim 35 characterized by the thermogravimetric analysis curve of FIG. 20.

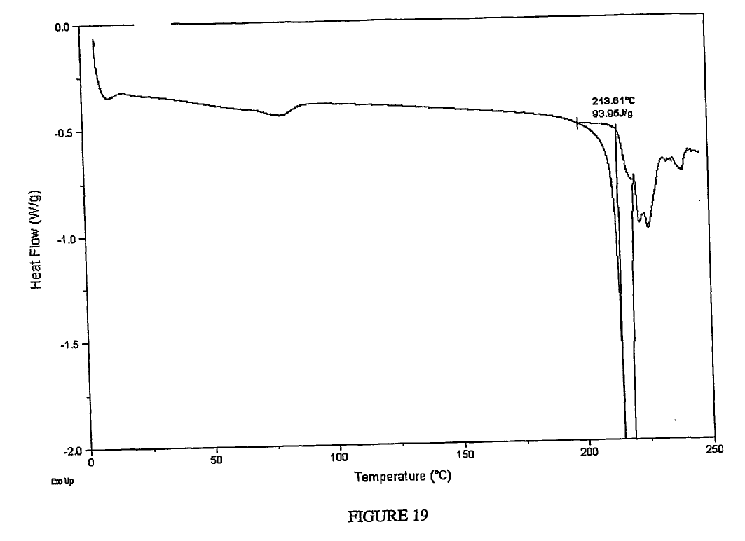

45. The crystalline ethanolate of claim 35 characterized by the differential scanning calorimetric (DSC) curve of FIG. 19.

46. A drug substance which is the dihydrogenphosphate salt of (2R)-4-oxo-4-[3-(trifluoromethyl)-5,6-dihydro[ 1,2,4]triazolo[4,3-π]pyrazin-7(8H)-yl]-1-(2,4,5-trifluorophenyl)butan-2-amine of structural formula I: 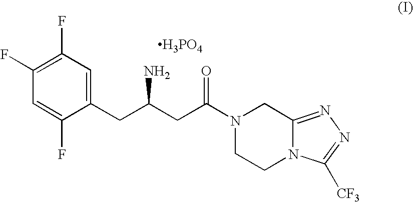
comprising a mixture of crystalline anhydrate Form I and crystalline anhydrate Form III.

47. A dihydrogenphosphate salt of (2R)-4-oxo-4-[3-(trifluoromethyl)-5,6-dihydro[ 1,2,4]triazolo[4,3-α]pyrazin-7(8H)-yl]- 1 -(2,4,5-trifluorophenyl)butan-2-amine of structural formula I: 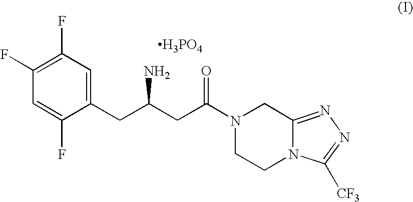
comprising a detectable amount of crystalline anhydrate Form I or crystalline anhydrate Form III or a mixture thereof.

48. A dihydrogenphosphate salt of (2R)-4-oxo-4-[3-(trifluoromethyl)-5,6-dihydro[ 1,2,4]triazolo[4,3-α]pyrazin-7(8H)-yl]- 1 -(2,4,5-trifluorophenyl)butan-2-amine of structural formula I: 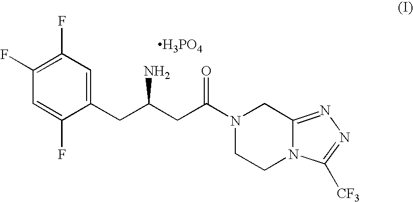
comprising substantially all by weight of crystalline anhydrate Form I or crystalline anhydrate Form III or a mixture thereof.

49. A pharmaceutical composition comprising a therapeutically effective amount of the salt of claim 1 or claim 12 or a mixture thereof in association with one or more pharmaceutically acceptable carriers or excipients.

50. A method of treating Type 2 diabetes comprising administering to a patient in need of such treatment a therapeutically effective amount of the salt according to claim 1 or claim 12 or a mixture thereof.

51-52. (canceled)
